(19) United States
(12) Patent Application Publication
(10) Pub. No.: US 2007/0196515 A1
(43) Pub. Date: Aug. 23, 2007

(54) Methods and compositions for improving cardiovascular risk factors and metabolic risk factors that cause syndrome X

(76) Inventor: Shil C. Kothari, Fairfield, CA (US)

Correspondence Address: GREENBERG TRAURIG LLP (LA), 2450 COLORADO AVENUE, SUITE 400E, INTELLECTUAL PROPERTY DEPARTMENT, SANTA MONICA, CA, 90404, US

(21) Appl. No.: 11/360,559

(22) Filed: Feb. 22, 2006

(51) Int. Cl. A61K 36/185

(52) U.S. Cl. 424/725

(57) ABSTRACT

The present invention provides for compositions and related methods using extracts from the Cissus quadrangularis plant to improve and eliminate various cardiovascular risk factors and metabolic risk factors that cause Syndrome X. Controlled university studies demonstrated that 300 mg Cissus quadrangularis plant extract provided to obese humans daily for six weeks was effective to provide numerous health benefits including weight loss, improving the bodies fat burning mechanisms, increasing serum serotonin levels which causes appetite suppression and prevents binge eating and/or emotional eating, reducing total cholesterol, LDL cholesterol, triglyceride levels, fasting blood sugar levels and blood pressure, and increasing HDL or “good” cholesterol levels. Additional university controlled experiments also showed that extracts from the Cissus quadrangularis plant were effective in increasing lipase inhibition, α-amylase inhibition, α-glucosidase inhibition in rodents and that large doses of Cissus quadrangularis plant extracts were not toxic to rodents.

### BACKGROUND OF THE INVENTION

1. Field of the Invention

The present invention relates generally to compositions and related methods using extracts from the Cissus quadrangularis plant to improve various cardiovascular risk factors and metabolic risk factors that are known to cause Syndrome X and provide a variety of related health benefits.

2. Description of the Related Art

Syndrome X (or metabolic syndrome) is a well-known syndrome, which is often defined by having obesity, cardiovascular disease and insulin resistance or diabetes. The American Heart Association (AHA) has indicated that Syndrome X in humans is characterized by a group of metabolic risk factors. More specifically, the AHA identified the following metabolic risk factors as contributing to Syndrome X:
1. Abdominal obesity (excessive fat tissue in and around the abdomen)
2. Atherogenic dyslipidemia (blood fat disorders—high triglycerides, low HDL cholesterol and high LDL cholesterol—that foster plaque buildups in artery walls)
3. Elevated blood pressure
4. Insulin resistance or glucose intolerance (the body can't properly use insulin or blood sugar)
5. Prothrombotic state (e.g., high fibrinogen or plasminogen activator inhibitor-1 in the blood)
6. Proinflammatory state (e.g., elevated C-reactive protein in the blood)

(See http://www.americanheart.org/presenter.jhtml?identifier=4756). The AHA and others have also acknowledged that people with Syndrome X have an increased risk of coronary heart disease and other diseases related to plaque buildups in artery walls (e.g., stroke and peripheral vascular disease) and type 2 diabetes. Unfortunately, Syndrome X has become increasingly common in the United States. The AHA has estimated that about 20-25 percent of US adults (over 50 million Americans) have Syndrome X.

The AHA also has indicated that the dominant underlying risk factors for Syndrome X appear to be abdominal obesity and insulin resistance. Insulin resistance is a generalized metabolic disorder, in which the body can't use insulin efficiently. This is why Syndrome X is also called insulin resistance syndrome. Other metabolic risk factors for Syndrome X include physical inactivity, aging and hormonal imbalance.

Unfortunately, some people are genetically predisposed to insulin resistance. It is well accepted that acquired metabolic risk factors, such as excess body fat and physical inactivity, can elicit insulin resistance and result in Syndrome X in these people. For individuals that are genetically predisposed to insulin resistance and, as a result, predisposed to acquiring Syndrome X, it is especially important to improve, control or eliminate the metabolic risk factors thought to cause insulin resistance and Syndrome X. It is believed that most people with insulin resistance have abdominal obesity. Accordingly, it is believed that abdominal obesity may play a larger role in causing insulin resistance and Syndrome X than other metabolic risk factors. It is well known that abdominal obesity can be controlled or reduced by overall weight loss and reduction of BMI.

Unfortunately, at the present, the biologic mechanisms of insulin resistance at the molecular level and how certain metabolic risk factors act to cause insulin resistance and Syndrome X aren't fully understood and appear to be complex. Similarly, at the present, the biological mechanisms of Syndrome X are also complex and not fully understood.

Presently, there is no universally-accepted criteria for diagnosing the Syndrome X. The AHA acknowledges that the criteria proposed by the National Cholesterol Education Program (NCEP) Adult Treatment Panel III (ATP III), are the most current and widely used criteria for diagnosing Syndrome X.

Additionally, the AHA and the National Heart, Lung, and Blood Institute recommend that the Syndrome X or metabolic syndrome be identified or diagnosed by the presence of three or more of the following metabolic risk factors:
1. Central obesity as measured by an elevated waist circumference:
   Men—Equal to or greater than 40 inches (102 cm)
   Women—Equal to or greater than 35 inches (88 cm)
2. Elevated triglycerides:
   Equal to or greater than 150 mg/dL
   Reduced HDL (“good”) cholesterol:
      Men—Less than 40 mg/dL
      Women—Less than 50 mg/dL
3. Elevated Blood pressure:
   Equal to or greater than 130/85 mm/Hg
4. Elevated fasting glucose:
   Equal to or greater than 100 mg/dL

(See http://www.americanheart.org/presenter.jhtml?identifier=534). Accordingly, control and improvement of these and related metabolic risk factor prevent the onset and treat Syndrome X. The AHA has indicated that to gain the most benefit from modifying multiple metabolic risk factors that cause Syndrome X, the underlying insulin resistant state must become a target of therapy and that the safest, most effective and preferred way to reduce insulin resistance in overweight and obese people is through weight loss and increased physical activity. The AHA has also indicated that other steps for managing Syndrome X that are also important for patients and their doctors include:
1. Routinely monitoring body weight (especially the index for central obesity), blood glucose, lipoproteins and blood pressure.
2. Treating individual risk factors (hyperlipidemia, hypertension and high blood glucose) according to established guidelines.
3. Carefully choosing anti-hypertensive drugs because different agents have different effects on insulin sensitivity.

(See http://www.americanheart.org/presenter.jhtml?identifier=534). Accordingly, improving and thereby eliminating these and related metabolic risk factors that cause Syndrome X, is an effective way of preventing, treating and controlling the progression of Syndrome X.

A related condition that effects most Americans is cardiovascular disease. In fact, cardiovascular disorders are the number one killer of both men and women in the United States. Coronary heart disease is so prevalent that it is estimated that one in five Americans have some form of it. It is also estimated that as many as 1.1 million Americans will have a coronary attack this year and about one-third of them will die from that attack.

The AHA has acknowledged that common cardiovascular risk factors for coronary heart disease and stroke that can be controlled or treated include, high total cholesterol (240 mg/dL or higher), high triglyceride levels (blood fats, 150 mg/dL or higher), high LDL cholesterol levels (greater than 100 mg/dL), low HDL cholesterol levels (less than 40 mg/dL for men; less than 50 mg/dL for women), high blood pressure (135/85 mm/Hg or higher), smoking, diabetes, physical inactivity and being overweight (BMI of 25.1 to 30.0) or obese (BMI of 30.0 or greater). (See http://strokeassociation.org/presenter.jhtml?identifier=3027394 & http://strokeassociation.org/presenter.jhtml?identifier=4716 which are incorporated herein by reference). It is well accepted that these cardiovascular risk factors cause cardiovascular disease and death. Accordingly, in an effort to prevent or treat cardiovascular disease and prolong death, improvement and elimination of these and related cardiovascular risk factors is highly desirable.

Various treatments have been created targeting improving or eliminating the treatable cardiovascular risk factors that cause various coronary ailments and the metabolic risk factors that cause Syndrome X. Unfortunately, none of these treatments have been shown to be particularly effective at improving or eliminating either the cardiovascular risk factors (elevated total cholesterol, triglyceride, LDL cholesterol, blood pressure, reduced HDL cholesterol, diabetes and being overweight or obese) or the metabolic risk factors (obesity, elevated triglycerides, fasting glucose levels and blood pressure and reduced HDL) that cause Syndrome X to such a degree that they are significantly improved or eliminated and, in the case of the metabolic risk factors, Syndrome X is prevented or treated. Additionally, the known treatments can often be difficult to administer, cause serious and undesirable side effects and often become less and less effective over time.

Accordingly, compositions and related methods that improve or eliminate well known cardiovascular risk factors and the metabolic risk factors that cause Syndrome X are needed. Additionally, compositions and related methods that providing additional health benefits, including but not limited to, increasing or improving fat burning mechanisms, providing appetite suppression and preventing binge eating and emotional eating, increasing lipase, α-amylase, α-glucosidase and trypsin inhibition thereby blocking the absorption of unwanted dietary fats, carbohydrates, sugars and protein, respectively, are needed. The present invention provides these and other related advantages.

### SUMMARY OF THE INVENTION

Briefly, and in general terms, by way of example and not limitation, one aspect of the present invention resides in improved compositions and related methods using extracts from the Cissus quadrangularis plant to improve or eliminate cardiovascular risk factors and the metabolic risk factors that cause Syndrome X, thereby preventing or treating Syndrome X. Additionally, by way of example and not limitation, another aspect of the present invention resides in using Cissus quadrangularis plant extracts to improve or eliminate at least one cardiovascular risk factor or metabolic risk factors that causes Syndrome X.

By way of example and not limitation, yet another aspect of the present invention relates to providing a mammal with an effective amount of Cissus quadrangularis extracts to provide the mammal with one or more of the following benefits (1) increased weight loss and reduced BMI, (2) increased or improved fat burning mechanisms, (3) appetite suppression and prevent binge eating and/or emotional eating, (4) increased lipase inhibition thereby blocking the absorption of unwanted dietary fats, (5) increased α-amylase inhibition thereby blocking the absorption of unwanted carbohydrates, (6) increased α-glucosidase inhibition thereby blocking the absorption of unwanted sugars, (7) increased trypsin inhibition thereby blocking the adsorption of unwanted protein, (8) reduced total cholesterol, LDL cholesterol, triglyceride levels or blood pressure, (9) increased HDL or “good” cholesterol levels, (10) reduced fasting blood sugar levels, (11) increased creatinine levels and (12) prevention or treatment of Syndrome X.

By way of example and not limitation, yet another aspect of the present invention relates to using a composition containing an effective amount of extracts from one or more Cissus quadrangularis plants to improve one or more cardiovascular risk factors in a mammal.

By way of example and not limitation, in yet another detailed aspect of the present invention, the effective amount of the Cissus quadrangularis plant extracts used in a composition and provided to a mammal is preferably 100 mg to 900 mg, more preferably 200 mg to 400 mg and most preferably 300 mg daily.

By way of example and not limitation, in yet another detailed aspect of the present invention, the Cissus quadrangularis plant extracts of a composition improve and thereby can eliminate one or more of the following cardiovascular risk factors in a mammal: (1) overweight or obese, (2) high total cholesterol, (3) high LDL cholesterol, (4) low HDL cholesterol, (5) high blood pressure, (6) high triglyceride levels, and (7) high fasting blood glucose levels.

By way of example and not limitation, in yet another detailed aspect of the present invention, Cissus quadrangularis plant extracts of a composition are used to improve or eliminate cardiovascular risk factors in a mammal by decreasing weight and BMI, lowering total cholesterol levels, decreasing LDL cholesterol levels, increasing HDL cholesterol levels, decreasing overall blood pressure, lowering triglyceride levels; lowering fasting blood glucose levels, increasing serum serotonin levels and increasing creatinine levels.

By way of example and not limitation, in yet another detailed aspect of the present invention, improving or eliminating the cardiovascular risk factors using Cissus quadrangularis plant extracts prevents or treats Syndrome X.

By way of example and not limitation, in yet another detailed aspect of the present invention, improving or eliminating the metabolic risk factors that cause Syndrome X using Cissus quadrangularis plant extracts prevents or treats Syndrome X.

By way of example and not limitation, in yet another detailed aspect of the present invention, a composition containing Cissus quadrangularis plant extracts is provided to a mammal to improve or eliminate one or more cardiovascular risk factors and a claim is made that the composition improves or eliminates one or more cardiovascular risk factors in a mammal.

By way of example and not limitation, in yet another detailed aspect of the present invention, extracts of Cissus quadrangularis plants are used to improve cardiovascular risk factors by reducing weight, total cholesterol, LDL cholesterol, blood pressure; triglyceride levels; and fasting blood glucose levels and increasing HDL cholesterol levels, serum serotonin levels and creatinine levels in a mammal.

By way of example and not limitation, in yet another detailed aspect of the present invention, extracts of Cissus quadrangularis plants are used to improve one or more metabolic risk factors that cause Syndrome X in a mammal.

By way of example and not limitation, in yet another detailed aspect of the present invention, extracts of Cissus quadrangularis plants are used to reduce excess weight or obesity, lower high total cholesterol and high LDL cholesterol, raise low HDL cholesterol, lower high blood pressure, high triglyceride levels, and high fasting blood glucose levels.

By way of example and not limitation, in yet another detailed aspect of the present invention, extracts of Cissus quadrangularis plants are used to improve metabolic risk factors by decreasing weight and BMI, lowering total cholesterol levels, decreasing LDL cholesterol levels, increasing HDL cholesterol levels, decreasing overall blood pressure, lowering triglyceride levels; and lowering fasting blood glucose levels.

In yet another detailed aspect of the present invention, using the extracts of Cissus quadrangularis plants to improve or eliminate metabolic risk factors of Syndrome X, treats or prevents the onset of Syndrome X.

In yet another detailed aspect of the present invention, Cissus quadrangularis plant extracts of a composition are provided to a mammal to improve or eliminate one or more metabolic risk factors that cause Syndrome X and a claim that the composition or the Cissus quadrangularis plants extracts of the composition improves or eliminates Syndrome X or one or more of the metabolic risk factors that cause Syndrome X in a mammal is made.

In yet another detailed aspect of the present invention, Cissus quadrangularis plant extracts of a composition are used to reduce weight, total cholesterol, LDL cholesterol, blood pressure, triglyceride levels, and fasting blood glucose levels and increase HDL cholesterol levels, serum serotonin levels and urinary fat metabolites in a mammal.

By way of example and not limitation, in yet another detailed aspect of the present invention, Cissus quadrangularis plant extracts of a composition are used to improve metabolic risk factors that cause Syndrome X by reducing weight, decreasing total cholesterol levels, decreasing LDL cholesterol levels, increasing HDL cholesterol levels, decreasing blood pressure, decreasing triglyceride levels and decreasing fasting blood glucose levels in a mammal.

### BRIEF DESCRIPTION OF THE DRAWINGS

FIG. 1 shows two bar graphs and their accompanying data charts demonstrate the results of an experiment measuring the % decrease in BMI and decrease in weight that resulted when a group of humans received six weeks of 300 mg/day Cissus quadrangularis extract supplementation or a placebo.

FIG. 2 is a bar graph demonstrating the kinetic and endpoint lipase inhibition activities of 10 mg/ml Cissus quadrangularis extract and 10 mg/ml Orlistat as a percentage.

FIG. 3 is a bar graph demonstrating the kinetic and endpoint \acute{α}-amylase inhibition activity of 10 mg/ml Cissus quadrangularis extract and 10 mg/ml Acarbose as a percentage.

FIG. 4 is a bar graph demonstrating the endpoint \acute{α}-glucosidase inhibition of 1 mg/ml of Cissus quadrangularis extract and 1 mg/ml Naringenin as a percentage.

FIG. 5 is a bar graph demonstrating the endpoint trypsin inhibition of 1 mg/ml Cissus quadrangularis extract and 1 mg/ml soy bean trypsin inhibitor as a percentage.

FIG. 6 is a chart summarizing the inhibition activity of Cissus quadrangularis extract as compared to other well known inhibiting compounds.

FIG. 7 is a bar graph demonstrating the results of an experiment measuring the average % increase in urinary fat metabolite malondialdehyde that resulted when a group of humans received six weeks of 300 mg/day Cissus quadrangularis extract supplementation or a placebo.

FIG. 8 is a bar graph and accompanying data chart demonstrating the results of an experiment measuring the average % decrease in fasting blood glucose that resulted when a group of humans received six weeks of 300 mg/day Cissus quadrangularis extract supplementation or a placebo.

FIG. 9 is a bar graph and accompanying data chart demonstrating the results of an experiment measuring the average % increase in serum serotonin that resulted when a group of humans received six weeks of 300 mg/day Cissus quadrangularis extract supplementation or a placebo.

FIG. 10 is a bar graph and accompanying data chart demonstrating the results of an experiment measuring the average % change in total cholesterol that resulted when a group of humans received six weeks of 300 mg/day Cissus quadrangularis extract supplementation or a placebo.

FIG. 11 is a bar graph and accompanying data chart demonstrating the results of an experiment measuring the average % decrease in triglycerides that resulted when a group of humans received six weeks of 300 mg/day Cissus quadrangularis extract supplementation or a placebo.

FIG. 12 is a bar graph and accompanying data chart demonstrating the results of an experiment measuring the average % decrease in LDL cholesterol that resulted when a group of humans received six weeks of 300 mg/day Cissus quadrangularis extract supplementation or a placebo.

FIG. 13 is a bar graph and accompanying data chart demonstrating the results of an experiment measuring the average % change in HDL cholesterol that resulted when a group of humans received six weeks of 300 mg/day Cissus quadrangularis extract supplementation or a placebo.

FIG. 14 is a bar graph and accompanying data chart demonstrating the results of an experiment measuring the average % increase in creatinine levels that resulted when a group of humans received six weeks of 300 mg/day Cissus quadrangularis extract supplementation or a placebo.

FIG. 15 is a summary chart demonstrating the results of an experiment measuring the effect on various cardiovascular and metabolic risk factors that resulted when a group of humans received six weeks of 300 mg/day Cissus quadrangularis extract supplementation or a placebo.

FIG. 16 is a bar graph demonstrating the results of an experiment measuring the % change in weight when doses of 8 g/kg and 12 g/kg of Cissus quadrangularis extract were given to rodents for two weeks to determine acute toxicity.

FIG. 17 is a chart demonstrating the results of an experiment measuring the effect 4 g/kg and 8 g/kg Cissus quadrangularis extract supplementation in rodents had on various biochemical parameters that determine sub-acute toxicity.

FIG. 18 are two charts demonstrating the results of experiments measuring the relative weight of organs harvested from male and female rodents supplemented with 8 g/kg Cissus quadrangularis extract and a control group to determine sub-acute histopathology.

FIG. 19 is a chart demonstrating the results of an experiment measuring the average decrease in systolic blood pressure that results when a group of humans received six weeks of 300 mg/day Cissus quadrangularis extract supplementation or a placebo

### DETAILED DESCRIPTION OF THE PREFERRED EMBODIMENTS

Cissus quadrangularis is an ancient medicinal plant of the Vitaceae family native to the hotter parts of Ceylon and India. It has been used for centuries for specific medicinal and culinary purposes. More specifically, it was prescribed in the ancient Ayurvedic texts as a general tonic and analgesic, with specific bone fracture healing properties. Modern research has shed some light on Cissus quadrangularis's ability to speed bone healing by showing that it acts as a glucocorticoid antagonist. Since anabolic/androgenic compounds are well known to act as antagonists to the glucocorticoid receptor as well as promote bone growth and fracture healing, it has been postulated that Cissus quadrangularis possesses anabolic and/or androgenic properties.

In addition to speeding the remodeling process of the healing bone, it is believed that Cissus quadrangularis also leads to a much faster increase in bone tensile strength. Clinical trial research has indicated that effective amounts of Cissus quadrangularis reduced fracture healing times by 55% to 33% when compared to controls. Additional evidence that Cissus quadrangularis exerts antiglucocorticoid properties is suggested by a number of studies where bones were weekend by treatment with cortisol, and upon administration of an effective amount of Cissus quadrangularis, the cortisol induced weakening was halted, and the healing process begun.

While studies have shown that the increased rate of bone healing may be of great significance to persons suffering from chronic diseases like osteoporosis, the antiglucocorticoid properties of Cissus quadrangularis are likely of much more interest to the average bodybuilder or athlete, since endogenous glucocorticoids, particularly cortisol, are not only catabolic to bone, but catabolize muscle tissue as well. Numerous studies over the years have suggested that glucorticoids, including the body's endogenous hormone cortisol activate pathways that degrade not only bone, but skeletal muscle tissue as well. One published report documented how glucocorticoids (including cortisol) induce muscle breakdown by activating the so-called ubiquitin-proteasome pathway of proteolysis. This pathway of tissue breakdown is important for removing damaged and non-functional proteins. However, when it is overactive during periods of elevated cortisol (e.g. disease states, stress, and overtraining) excess amounts of normal tissue are broken down as well. By exerting an anabolic, antiglucorticoid effect, Cissus quadrangularis could help to preserve muscle tissue during times of physical and emotional stress.

In an effort to find compositions and related methods that improve or eliminate cardiovascular risk factors and metabolic risk factors that cause Syndrome X, we investigated the effects of compositions containing Cissus quadrangularis extracts on a variety of cardiovascular and metabolic risk factors and other related health factors. In particular, we investigated the effect non-toxic amounts of a Cissus quadrangularis extract have on (1) weight and BMI, (2) fat burning mechanisms, (3) appetite suppression and binge eating and/or emotional eating, (4) lipase, α-amylase, α-glucosidase and trypsin inhibition (4) total cholesterol, LDL cholesterol, HDL cholesterol and triglyceride levels and blood pressure, (5) fasting blood sugar in mammals, and (6) creatinine levels, all of which are cardiovascular or metabolic risk factors or provide other health related benefits.

Additionally, to ensure that the compositions of Cissus quadrangularis plant extracts we tested were safe in mammals, we tested the toxicity of comparatively very large doses of the Cissus quadrangularis plant extracts in rodent models. In particular, we preformed university controlled double blind studies of acute and sub-acute toxicity of large doses of Cissus quadrangularis extracts which showed that the compositions and related methods of the claimed invention were not toxic in rodent models and would be safe for human consumption.

While methods of obtaining extracts of from a plant like Cissus quadrangularis are common and well known, the Cissus quadrangularis extracts for all the experiments and research finding below were obtained by The extraction process is an aqueous water extract of the aerial portion of the plant that uses an extract: dried plant ratio of 1:4 to 1:15 depending on the seasonal harvesting of the Cissus plant. The process uses a live steam for sterilization and further purification of the active components. The extract is then either spray dried or drum dried to produce a standardized extract containing no less then 2.5% keto-steroids, verified by high performance liquid chromatography (HPLC) using a ultraviolet/visible light (UV/VIS) detector.

The following is a summary of the experiments and research findings relating to compositions and methods of the present invention. The methods and compositions of the present invention (1) prevent and control Syndrome X, (2) increase weight loss and reduce BMI, (3) improve fat burning by increasing excretion of urinary fat metabolite, malondialdehyde, (4) increase serum serotonin levels, which provides the benefit of appetite suppression and prevention of binge eating and/or emotional eating. (5) increase lipase inhibition thereby blocking the absorption of unwanted dietary fats, (5) increase α-amylase inhibition thereby blocking the absorption of unwanted carbohydrates, (6) increase α-glucosidase inhibition thereby blocking the absorption of unwanted sugars, (7) reduce the total cholesterol levels in obese subjects with healthy cholesterol levels, (8) reduce LDL cholesterol levels in obese subjects with healthy cholesterol levels, (9) reduce triglyceride levels in obese subjects with healthy triglyceride levels, (10) increase HDL or “good” cholesterol levels in obese subjects with healthy, (11) reduce fasting blood sugar levels in obese subjects cholesterol levels (12) decrease blood pressure and (13) increasing creatinine levels. It should be appreciated that these and other benefits provided by the methods and compositions of the present invention aid in the prevention and control of the onset and progression of obesity, diabetes, coronary heart disease and Syndrome X. Syndrome X, an insulin-resistant condition, is thought to play a causative role in the induction of obesity, diabetes and heart disease.

Lipase Inhibition

To determine if Cissus quadrangularis extracts provide lipase inhibiting activity, and therefore blocks of the absorption of lipids (fats),we tested the kinetic and endpoint inhibition of 10 mg/ml aqueous and ethanolic extracts of Cissus quadrangularis. An MGT enzyme assay kit was used with the method described in the 1991 article by Liodakis, A., Drew, J., Chan, R., and Sawyer, entitled “Spectrofluorometric determination of lipase activity.” (Biochem Internat at 23(5): 825-834).

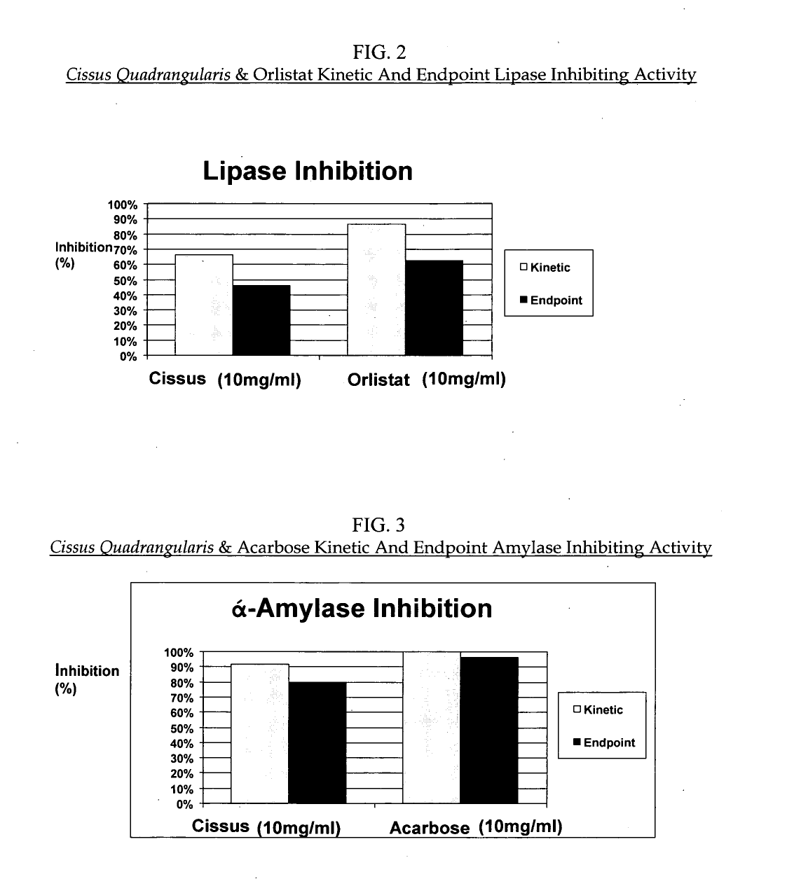

FIG. 2 shows the results of our experiment in the form of a bar graph which compares the lipase inhibiting activity of 10 mg/ml of Cissus quadrangularis extracts to that of 10 mg/ml of Orlistat (marketed by Roche as Zenical™), a well known and popular weight loss drug that acts as a lipase inhibitor. Cissus quadrangularis extracts provided significant kinetic and endpoint lipase inhibition. In it important to note that while Cissus quadrangularis extracts provided lipase inhibiting activity similar to that of Zenical, Cissus quadrangularis extracts do not to have any of the common side effects or drug interaction associated with Orlistat. Some of the side effects associated with Orlistat include acute infection of the nose, throat or sinus, fatty bowel movements, discharge of stools when passing gas, uncontrollable bowel movements, oily leakage from the rectum, oily stools, and interactions with drugs like cyclosporine. Accordingly, an effective amount of Cissus quadrangularis provides significant lipase inhibiting activity and prevents the adsorption of lipids, without causing the side effects commonly associated with lipase inhibiting drugs.

Amylase Inhibition

To determine if Cissus quadrangularis extracts provide α-amylase inhibiting activity, which blocks of the absorption of carbohydrates and helps manages blood sugar, we performed kinetic and endpoint inhibition studies of aqueous and ethanolic extracts of Cissus quadrangularis on the activity of α-amylase. α-Amylase (EC 3.2.1.1) (1,4-alpha-D-glucan glucanohydrolase), obtained from Sigma-Aldrich (A 4268) was used as reference, and represented 100% activity. The various fractions of Cissus quadrangularis were used to determine the percentage of inhibition activity. The methods described in 1978 Kikumoto article entitled “An improved assay method for amylase activity using an amylodextrin fraction as substrate” (Carbohydrate Research 61: 369-375) were used. FIG. 3 shows the results of our experiment in the form of a bar graph which compares the α-Amylase inhibiting activity of 10 mg/ml of Cissus quadrangularis extracts to 10 mg/ml of Acarbalose. The results shown in bar graph of FIG. 3 demonstrate that Cissus quadrangularis extracts provide significant α-Amylase inhibiting activity, similar to the α-Amylase inhibiting activity of Acarbalose.

Glucosidase Inhibition

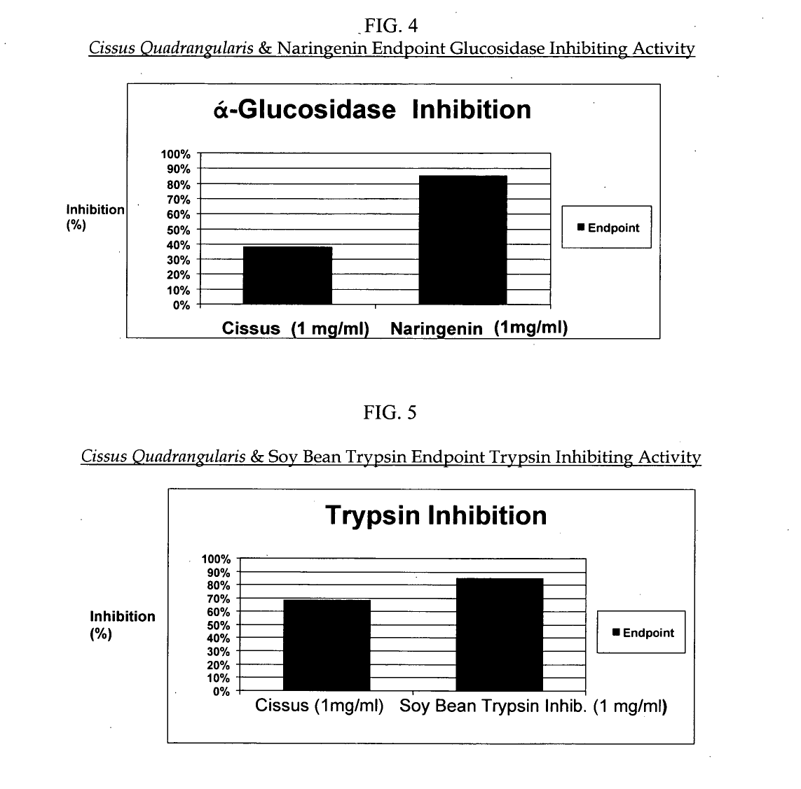

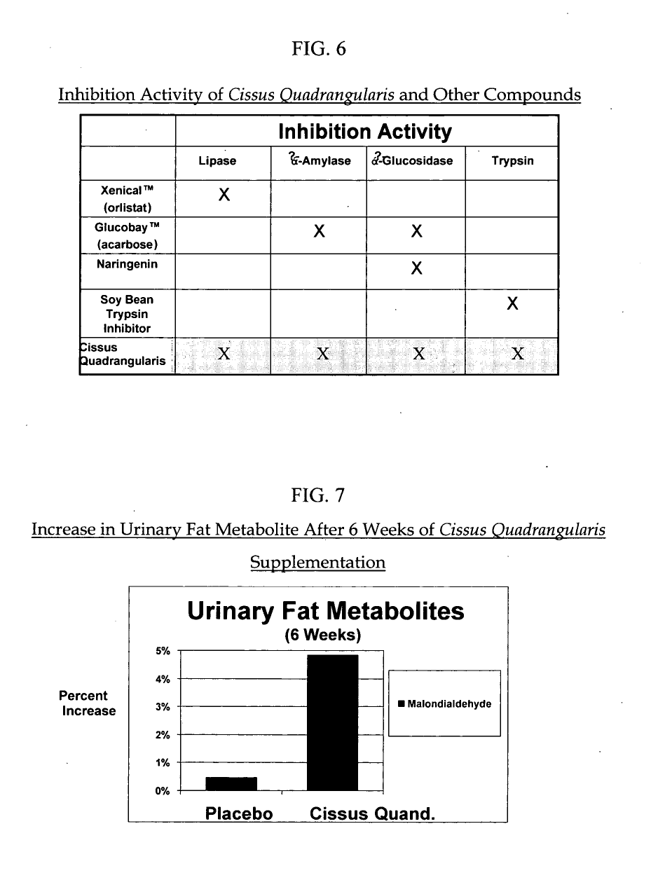

To determine if Cissus quadrangularis extracts provide α-glucosidase inhibiting activity, which blocks of the absorption of glucose (6-carbon sugar), we performed endpoint inhibition studies of aqueous and ethanolic extracts of Cissus quadrangularis on the activity of yeast α-glucosidase. α-Glucosidase (EC 3.2.1.1) catalyzes the hydrolysis of terminal 1,4-linked α-D-glucose residues successively from the non-reducing ends of maltooligo- and to a lesser extent polysacharides with release of β-D-glucose. Most forms of the enzyme can slowly hydrolyze 1,6-α-D-glucosidic bonds. As the results of the bar graph of FIG. 4 demonstrate, Cissus quadrangularis extracts showed significant α-glucosidase inhibiting activity indicating the blocking of the absorption of glucose. The activity of α-glucosidase was measured in the presence and absence of Cissus quadrangularis fractions, to determine the extent of inhibition of activity.

Trypsin Inhibition

To determine if Cissus quadrangularis extracts provide trypsin inhibiting activity, indicating prevention of the breakdown of protein at arginine and lysine residues, we performed endpoint inhibition studies of extracts of Cissus quadrangularis. Trypsin (EC 3.4.21.4) from bovine pancreas was obtained from Sigma-Aldrich (T 4665). Inhibition of activity was determined for the extracts of Cissus quadrangularis, with a trypsin inhibitor from Sigma-Aldrich (T 0256) used a standard inhibitor and continuous spectrophotometric rate determination to determine the inhibition.

FIG. 3 show that Cissus quadrangularis extracts inhibit the activity of both salivary and pancreatic α-amylase. The type of inhibition was found to be amylase-type specific, with salivary amylase being inhibited in a non-competitive manner (inhibitor and substrate bind simultaneously to enzyme molecule) while the inhibition of pancreatic amylase was an irreversible inhibition (it is believed that the inhibitor chemically modifies enzyme structure; modified enzyme functioning at a reduced rate). Accordingly, an effective amount of Cissus quadrangularis extract provides significant α-amylase inhibiting activity, which blocks of the absorption of carbohydrates and helps manages blood sugar.

Human Studies on Cardiovascular and Metabolic Risk Factors

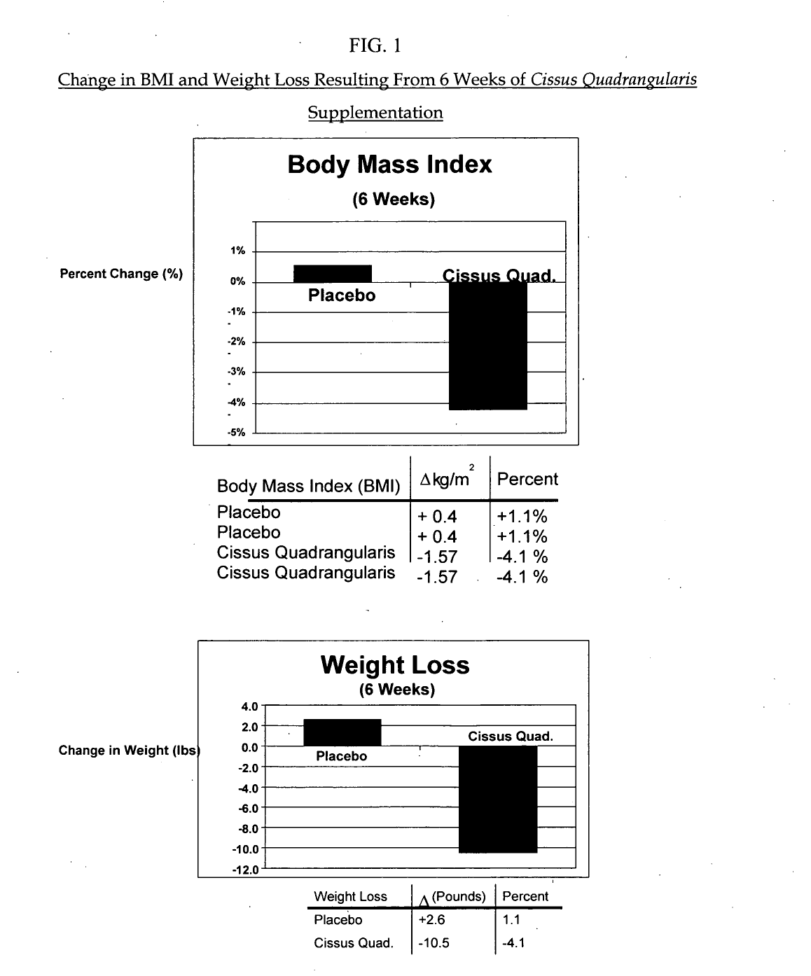

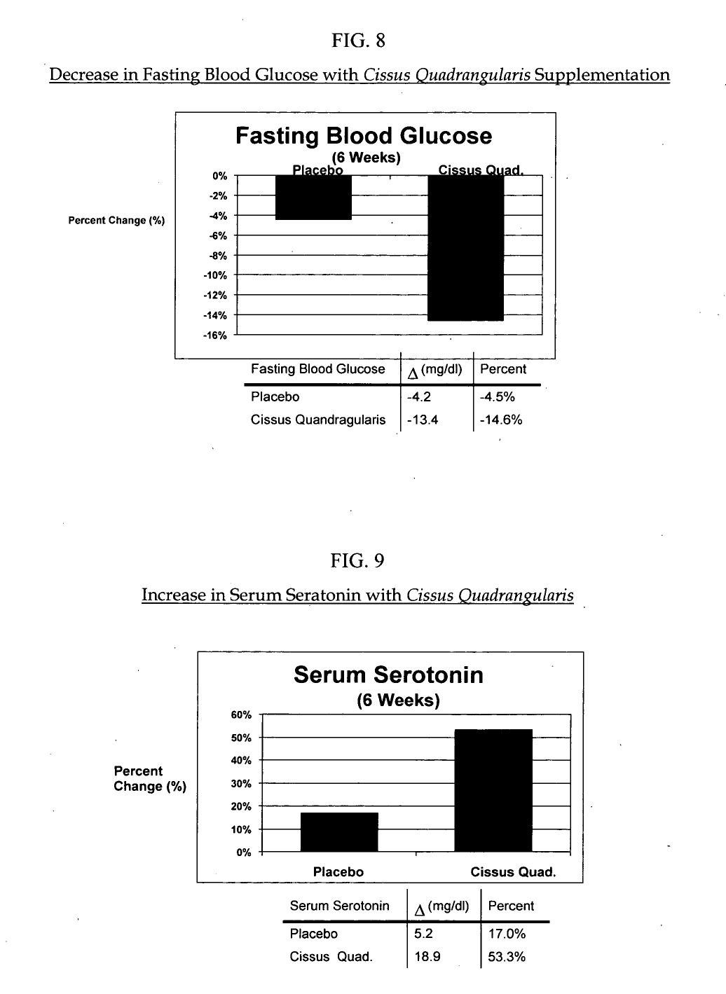

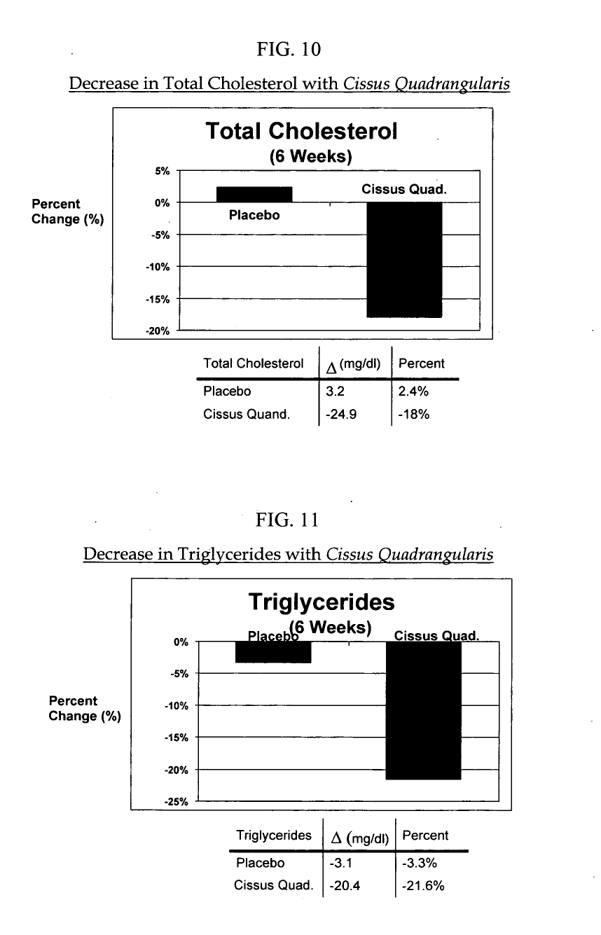

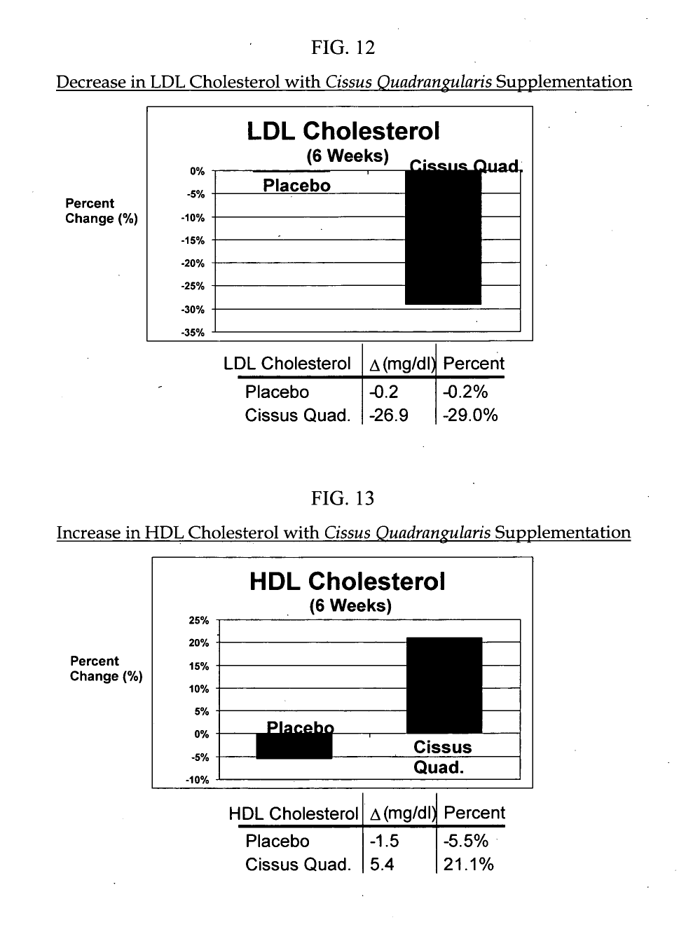

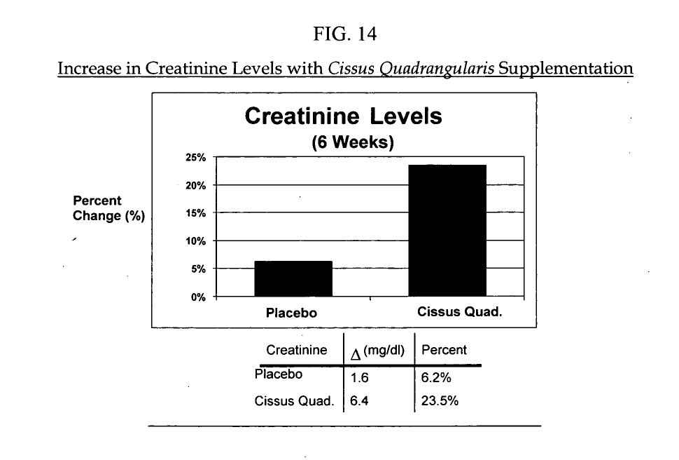

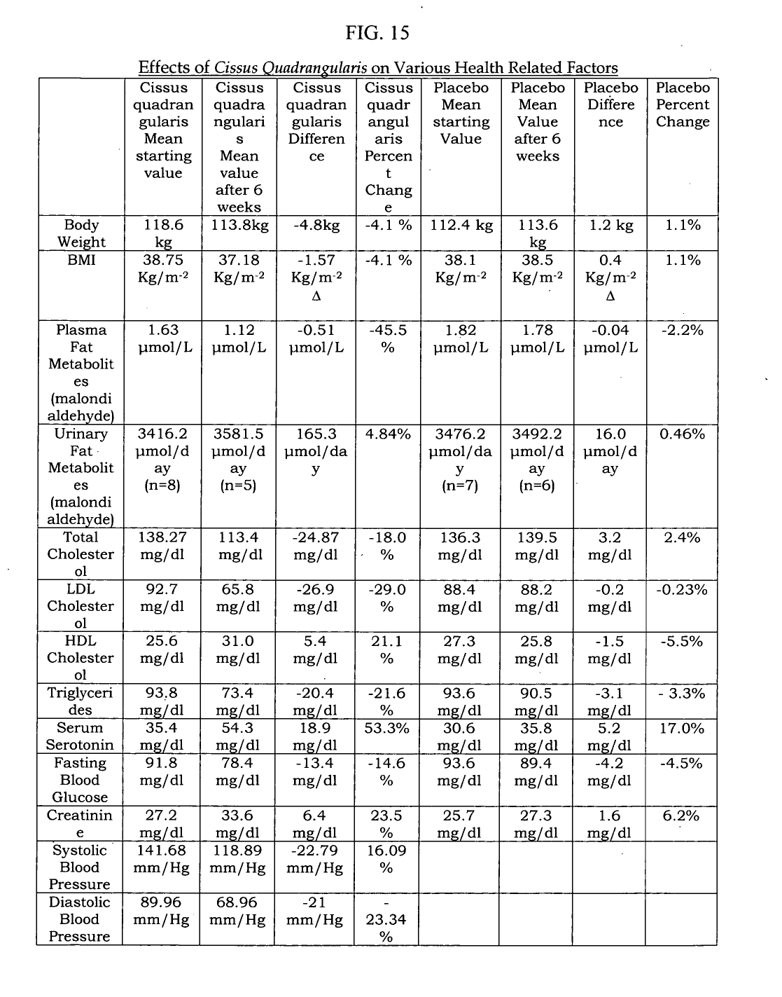

To determine the effect of Cissus quadrangularis plant extracts on humans, a university controlled, randomized, double-blind, placebo-controlled study was conducted at the University of Yaoundé I, Cameroon, Department of Biochemistry. More specifically, the experiments determined the effect of 300 mg of Cissus quadrangularis extract (administered in two 150 mg doses morning and evening) for six weeks, on the body weight, body mass index, fat loss, fat metabolites, blood lipids, fasting blood glucose, serum serotonin and creatinine of sixty moderately obese human subject. The human subject were not restricted in diet but asked to eat sensibly. Mild exercise was recommended but not monitored. The table shown in FIG. 15 shows the results of the experiments, which demonstrate that 300 mg of Cissus quadrangularis extract administered for six weeks improved, often significantly, body weight, body mass index, fat loss, fat metabolites, blood lipids, fasting blood glucose, serum serotonin and creatinine levels. FIGS. 1-2, 7-14 and 19 each show bar graphs (or a chart in the case of FIG. 19) with the results of Cissus quadrangularis supplementation study for each of the variables listed in the table of FIG. 15. Accordingly, it should be appreciated that 300 mg of Cissus quadrangularis extract can be used to improve cardiovascular risk factors and metabolic risk factors that cause Syndrome X and provide related health benefits.

Safety Studies

To determine if Cissus quadrangularis plant extracts are safe, we performed independent university studies on the toxicity of very large doses Cissus quadrangularis plant extract. The results of the research, which are described in detail below and shown in FIG. 17, showed no mortality or toxic effects. Additionally, because the LD50 of Cissus quadrangularis tested by oral administration in rats is more than 12 gm/kg body weight, or approximately 32,000 times the recommended dose for humans, the doses recommended for humans would not be toxic.

Acute Toxicity—LD50>12 g/kg with no Mortalities.

To ensure that large dosages of Cissus quadrangularis extract do not result in acute toxicity, acute toxicity experiments using the methods and protocols described on pages 185 of Hayes 1987 article entitled “Guidelines of Acute Oral Toxicity Testings” found in the In Principles and Methods of Toxicology, 2nd Edition (Raven Press Ltd,. New York), were preformed. More specifically, mice were divided into 3 groups of 6 mice each. Group 1 was the control group and groups two and three were the test groups. Group one, which served as the control group, was given daily oral doses of distilled water. The two test groups were each given daily oral doses of 8 g/kg or 12 g/kg powdered extract of Cissus quadrangularis dissolved in 0.5 ml of distilled water.

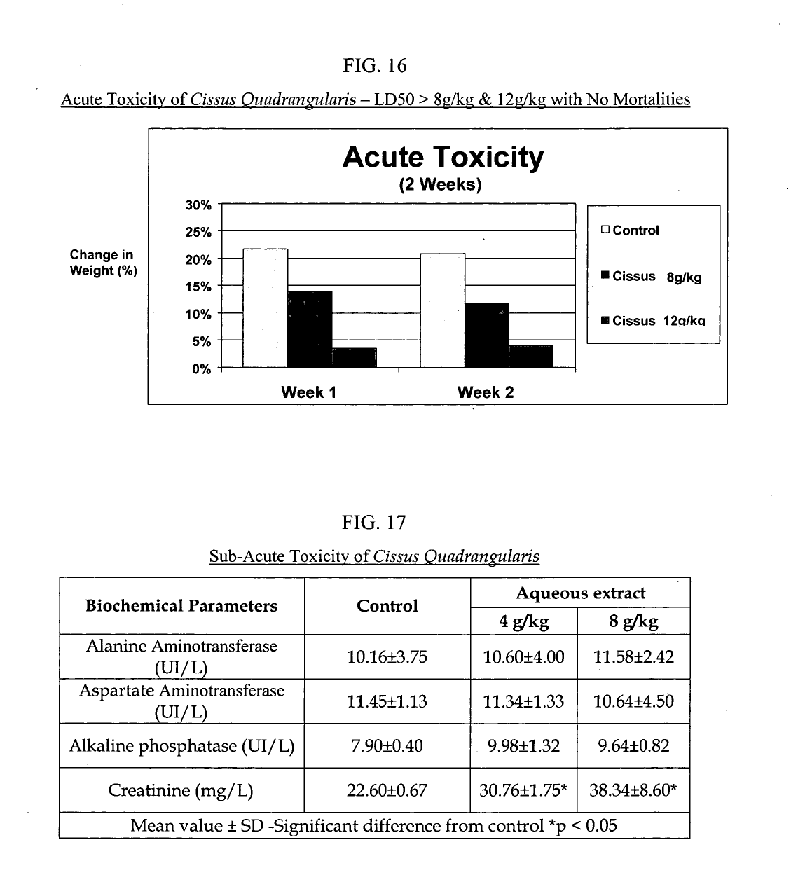

It should be appreciated that because the acute toxicology experiment was carried out for two weeks, they demonstrate that sub-acute toxicity is also not an issue at these levels. The study was carried for two full weeks to see if there would be any mortalities. The animals were observed on a continuous basis for one hour after ingestion of the Cissus quadrangularis extract and at half hourly intervals thereafter for the next seven hours, for any change in behavioral activity and then every twelve hours for the next 2 weeks for any mortality. The method described in the Miller and Tainter (1944) article entitled “Estimation of the LD50 and its error by means of Logarithmic-probit graph paper” (Proceedings of the Society for Experimental Biology And Medicine at 57:261-264) was used to determine the LD50. FIG. 16 shows a chart of the change in weight as a percentage for the two groups feed 8 g/kg and 12 g/kg of Cissus quadrangularis aqueous extract each and the control group.

Sub-Acute Toxicity

To ensure that high dosages Cissus quadrangularis do not result in sub-acute toxicity, specific sub-acute toxicity experiments were performed. More specifically, male and female Wistar Albino rats were divided into three groups of twelve rats each and labeled groups A, B & C. Group A served as the control group and was given distilled water throughout the experimental period of fourteen days. Group B and C served as the test groups and were given daily oral doses of Cissus quadrangularis (aqueous extract of 4 g/kg and 8 g/kg respectively) throughout the experimental period.

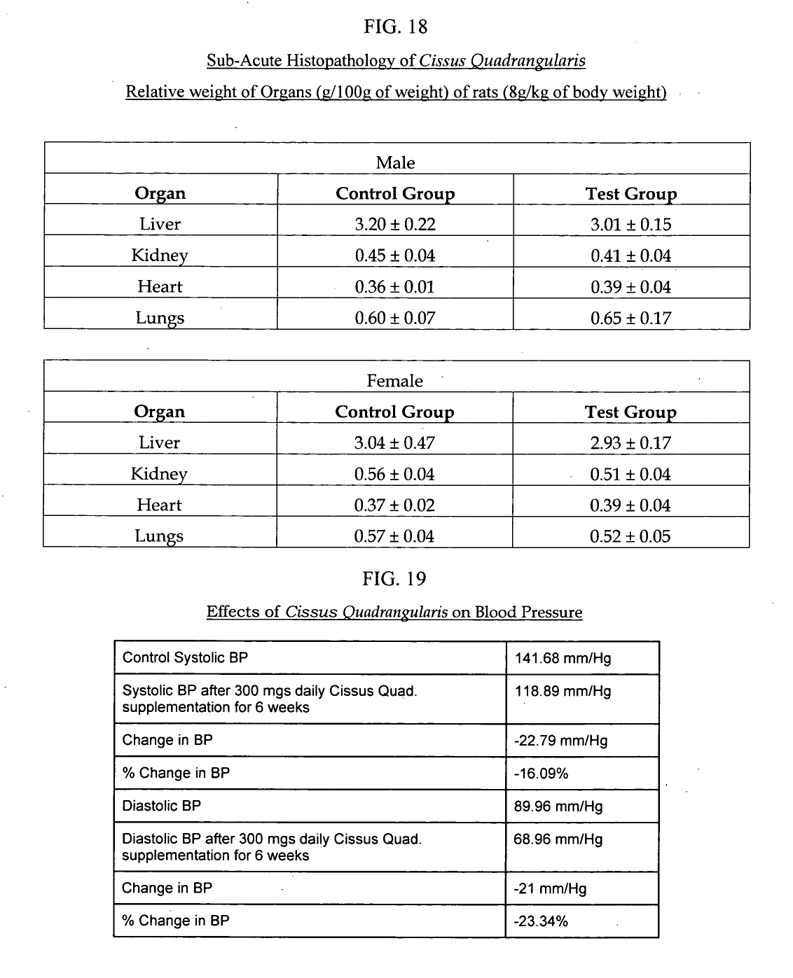

Six rats from each group were then sacrificed by cervical dislocation on day fourteen (sub-acute toxicity) and blood was collected by decapitation for biochemical assays. More specifically, tests to determine aspartate aminotransferase (AST) and alanine amino transferase (ALT) activity were performed using the methods described in the Reitman and Frankel 1957 article entitled “A colorimetric method for the determination of serum glutamate oxaloacetate and pyruvate transaminase” found in the American Journal of Clinical Pathology 28:56-63. Similarly, creatinine levels were measured using the method described in the Hare 1950 article entitled “Endogenous creatinine in serum and urine” found in the Proceedings of the Society for Experimental Biology and Medicine at 74: 148. Additionally, liver, kidney, heart and lung samples were collected in 10% formalin for histopathology studies using the methods described in “Techniques Histologiques: Masson et Cie éditeurs” 120, Boulevard Saint Germaine, Paris at pp 87, 128, 243 (Gabe 1968). Paraffin sections were stained with haematoxylin and eosin (H&E) and microscopically (Leitz Wetzlar Germany) and examined using a microscope at a magnification of 25×. FIG. 18 shows two summary charts of the histopathology studies results for the male and female rats separately, which show no significant differences between the control group and the experimental group.

Experimental data were analyzed by employing the analysis of variance (ANOVA). Duncan's multiple range test was used to determine significant differences between means. The statistical analysis system (SAS) package was used for this purpose.

FIG. 17 is chart summarizing the results of our biochemical assays which determined aspartate aminotransferase (ASAT) and alanine amino transferase (ALAT) activity As the summary chart of FIG. 17 demonstrates, at a dose of 8 g/kg Cissus quadrangularis, no sub-acute toxicity was found for alanine transaminase, aspartate transaminase, alkaline phosphatase or creatinine. FIG. 17 shows that the does of 8 g/kg Cissus quadrangularis resulted in a significant difference in creatinine levels, indicative of the muscle building effects of the keto-steroids in Cissus quadrangularis. The increased creatinine levels were not high enough to suggest toxicity. All other parameters of toxicity showed no significant change. In addition to these test results, Cissus quadrangularis's safety is further confirmed that it is listed in the U.S. Department of Agriculture's list of edible vegetables, its stems have been consumed for centuries throughout Asia and Africa as a culinary vegetable and it is believed that it presents no known drug interactions.

It should be appreciated that the compositions of the present invention and Cissus quadrangularis extracts generally can be used as a nutritional ingredient in a wide variety of dietary supplements and nutraceutical food and beverage products.

The foregoing detailed description of the present invention is provided for the purposes of illustration, and is not intended to be exhaustive or to limit the invention to the particular embodiments disclosed. The embodiments may provide different capabilities and benefits, depending on the configuration used to implement the key features of the invention. Accordingly, the scope of the present invention is defined only by the following claims.

What is claimed is:

1. A composition for improving one or more cardiovascular risk factors in a mammal comprising:
a composition containing an effective amount of extracts from one or more Cissus quadrangularis plants to improve one or more cardiovascular risk factors in a mammal.

2. A composition of claim 1, wherein the effective amount of the Cissus quadrangularis plant extracts in the composition provides 100 mg to 900 mg of Cissus quadrangularis plant extracts to the mammal daily.

3. A composition of claim 1, wherein the effective amount of the Cissus quadrangularis plant extracts in the composition provides 200 mg to 400 mg of Cissus quadrangularis plant extract to the mammal daily.

4. A composition of claim 1, wherein the effective amount of the Cissus quadrangularis plant extracts in the composition provides 300 mg of Cissus quadrangularis plant extract to the mammal daily.

5. A composition of claim 3, wherein the composition improves more than one of the following cardiovascular risk factors in the mammal:
a. weight or BMI;
b. total cholesterol;
c. LDL cholesterol;
d. HDL cholesterol;
e. blood pressure;
f. triglyceride levels; and
g. fasting blood glucose levels.

6. The composition of claim 3, wherein the composition improves cardiovascular risk factors by:
a. decreasing weight and BMI;
b. lowering total cholesterol levels;
c. decreasing LDL cholesterol levels;
d. increasing HDL cholesterol levels;
e. decreasing overall blood pressure;
f. lowering triglyceride levels; and
g. lowering fasting blood glucose levels.

7. The composition of claim 5, wherein improving one or more of the cardiovascular risk factors further treats Syndrome X or prevents the onset of Syndrome X.

8. The composition of claim 6, wherein the composition further treats Syndrome X or prevents the onset of Syndrome X by eliminating one or more metabolic risk factor that causes Syndrome X.

9. A method for improving or eliminating cardiovascular risk factors comprising:
a. providing a composition containing an effective amount of extracts from one or more Cissus quadrangularis plants to a mammal to improve or eliminate one or more cardiovascular risk factors; and
b. claiming that the composition improves or eliminates one or more cardiovascular risk factors in a mammal.

10. A method of claim 9, wherein the effective amount of Cissus quadrangularis plant extracts in the composition provided to the mammal is 100 g to 600 mg daily.

11. A method of claim 9, wherein the effective amount of the Cissus quadrangularis plant extracts in the composition provided to the mammal is 200 mg to 400 mg daily.

12. A method of claim 9, wherein the effective amount of the Cissus quadrangularis plant extracts in the composition provided to the mammal is 300 mg daily.

13. A method of claim 11, wherein extracts of the Cissus quadrangularis plants improves or eliminates cardiovascular risk factors by reducing weight, total cholesterol, LDL cholesterol, blood pressure; triglyceride levels; and fasting blood glucose levels and increasing HDL cholesterol levels in the mammal.

14. A method of claim 12, wherein the composition improves or eliminates at least three cardiovascular risk factors by causing three or more of the following to occur:
a. reduction of weight;
b. decrease in total cholesterol levels;
c. decrease in LDL cholesterol levels;
d. increase in HDL cholesterol levels;
e. decrease in blood pressure;
f. decrease in triglyceride levels; and
g. decrease in fasting blood glucose levels in a mammal
h. increasing urinary, fat metabolites; and
i. increasing serum serotonin levels.

15. A composition for improving or eliminating one or more of the metabolic risk factors that cause Syndrome X in a mammal comprising:
a composition containing an effective amount of extracts from one or more Cissus quadrangularis plants that improves or eliminates one or more metabolic risk factor that causes Syndrome X in a mammal.

16. A composition of claim 15, wherein the effective amount of the Cissus quadrangularis plant extracts in the composition provides 100 mg to 900 mg of Cissus quadrangularis plant extracts to the mammal daily.

17. A composition of claim 15, wherein the effective amount of the Cissus quadrangularis plant extracts in the composition provides 200 mg to 400 mg of Cissus quadrangularis plant extracts to the mammal daily.

18. A composition of claim 15, wherein the effective amount of the Cissus quadrangularis plant extracts in the composition provides 300 mg of Cissus quadrangularis plant extracts to the mammal daily.

19. The composition of claim 17, wherein the composition improves or eliminates more than one of the following metabolic risk factors in a mammal:
a. obesity;
b. high total cholesterol;
c. high LDL cholesterol;
d. low HDL cholesterol;
e. high blood pressure;
f. high triglyceride levels; and
g. high fasting blood glucose levels.

20. The composition of claim 17, wherein the composition improves or eliminates metabolic risk factors by:
a. decreasing weight and BMI;
b. lowering total cholesterol levels;
c. decreasing LDL cholesterol levels;
d. increasing HDL cholesterol levels;
e. decreasing overall blood pressure;
f. lowering triglyceride levels; and
g. lowering fasting blood glucose levels.

21. The composition of claim 19, wherein improving or eliminating one or more of the metabolic risk factors treats Syndrome X or prevents the onset of Syndrome X.

22. A method for improving or eliminating the metabolic risk factors that cause Syndrome X comprising:
a. providing a composition containing an effective amount of extracts of one or more Cissus quadrangularis plants to a mammal to improve or eliminate one or more metabolic risk factors that cause Syndrome X; and
b. claiming that the composition improves or eliminates Syndrome X or one or more of the metabolic risk factors that cause Syndrome X in a mammal.

23. A method of claim 22, wherein the effective amount of Cissus quadrangularis plant extracts in the composition provided to the mammal is 100 mg to 600 mg daily.

24. A method of claim 22, wherein the effective amount of the Cissus quadrangularis planet extracts in the composition provided to the mammal is 200 mg to 400 mg daily.

25. A method of claim 22, wherein the effective amount of the Cissus quadrangularis plant extracts in the composition provided to the mammal is 300 mg daily.

26. A method of claim 24, wherein the Cissus quadrangularis plant extracts reduce weight, total cholesterol, LDL cholesterol, blood pressure; triglyceride levels; and fasting blood glucose levels, and increase HDL cholesterol levels, serum serotonin levels and urinary fat metabolites in a mammal.

27. A method of claim 24, wherein the composition improves or eliminates at least three metabolic risk factors that cause Syndrome X by:
a. reducing weight;
b. decreasing total cholesterol levels;
c. decreasing LDL cholesterol levels;
d. increasing HDL cholesterol levels;
e. decreasing blood pressure;
f. decreasing triglyceride levels; and
g. decreasing fasting blood glucose levels in a mammal.

28. A composition for inhibiting any combination of lipase, α-amylase, α-glucosidase and trypsin in a mammal.

29. A method for inhibiting any combination of lipase, α-amylase, α-glucosidase and trypsin in a mammal comprising:
a. providing a composition containing an effective amount of extracts of one or more Cissus quadrangularis plants to a mammal to inhibiting any combination of lipase, α-amylase, α-glucosidase and trypsin in the mammal; and
b. claiming that the composition inhibits any combination of lipase, α-amylase, α-glucosidase and trypsin in a mammal.
